(19) United States
(12) Patent Application Publication
(10) Pub. No.: US 2014/0186458 A1
(43) Pub. Date: Jul. 3, 2014

(54) DIETARY BEVERAGE

(71) Applicants: Vyacheslav Nikolaevich Trifonov, Zarechni (RU); Julia Anatoljevn Elistratova, Penza (RU); Konstantin Gennadievich Elistratov, Penza (RU); Natalia Vyaheslavovna Kurus, Penza (RU); Irina Vladimirovna Homykova, Penza (RU); Tatiana Viktorovna Elistratova, Penza (RU)

(72) Inventors: Vyacheslav Nikolaevich Trifonov, Zarechni (RU); Julia Anatoljevn Elistratova, Penza (RU); Konstantin Gennadievich Elistratov, Penza (RU); Natalia Vyaheslavovna Kurus, Penza (RU); Irina Vladimirovna Homykova, Penza (RU); Tatiana Viktorovna Elistratova, Penza (RU)

(21) Appl. No.: 14/125,823

(22) PCT Filed: Jul. 3, 2012
(86) PCT No.: PCT/RU12/00527

§ 371 Date: Dec. 12, 2013

(30) Foreign Application Priority Data
Jul. 5, 2011 (RU) 2011128763

(51) Int. Cl. A61K 35/64; A23L 2/39; A23F 5/44; A61K 9/00

(52) U.S. Cl. CPC A61K 35/642; A61K 9/0053; A23L 2/39; A23F 5/44
USPC 424/539

(57) ABSTRACT

The invention relates to the food industry, in particular to biologically active dietary supplements (BADSs), and is intended for prophylaxis and therapeutic prophylaxis in conditions associated with fatigue, and is in the form of a coffee substitute or other beverage and consists of: 1 part drone brood, 3-9 parts glucose and/or fructose, and 1-100 parts fillers (coffee or another beverage in a powder form).

### CROSS-REFERENCE TO RELATED APPLICATIONS

This application is a U.S. national stage application of an international application PCT/RU2012/000527 filed on 3 Jul. 2012, published as WO2013006093, which international application claims priority of a Russian Federation patent application RU2011128763 filed on 5 Jul. 2011.

### FIELD OF THE INVENTION

The invention relates to the food industry and to medicine, more specifically to biologically active dietary supplements (BADS), and is intended for use in preventive and therapeutic treatment of health conditions associated with fatigue.

### BACKGROUND OF THE INVENTION

Currently, no drone brood-based coffee substitutes or other beverages with tonic properties are produced in Russia or abroad.

A “Biologically Active Supplement For The Manufacture of Cosmetic Products with Regenerative Activity” was patented in Russia (Pat. No. 2,128,501). This biologically active supplement comprises drone bee larvae recommended for use in the manufacture of cosmetic products with regenerative properties. A method for producing a biologically active product from drone bee larvae, or worker bees, or queen-bees (Pat. No. 74872/1980, OSIM) as a bioactive agent used in the manufacture of medicinal and cosmetic products, was patented in Romania. This product contains lactose that does not dissolve in water and forms a suspension. Also known is the Pat. No. 2,245,155—COMPOSITION OF PRESERVED BEE BROOD (DRONE BROOD AND QUEEN-BEE LARVAE) HOMOGENATE. This substance is viewed as a prototype here.

Larval homogenate represents an easily available, cheap and promising source for obtaining medicinal and food products. The neutral reaction of brood and its nutrient content obviously provide a favorable environment for the growth of bacteria and fungi, which makes the raw materials rapidly perishable under improper storage conditions. The addition of sorbic and citric acids to the prototype stabilizes drone brood; however, acids have an adverse effect on the biologically active constituents of drone brood, causing their destruction.

### PREFERRED EMBODIMENT OF THE INVENTION

It is proposed to preserve drone brood using glucose or fructose by mixing 1 part of drone brood homogenate with 3 to 9 parts of glucose and add this mixture to a dry coffee substitute or other beverage to be subsequently dissolved in water.

As was shown in experiments, this mixture is well stabilized in terms of its physicochemical parameters, which allows for long-term storage of this product (more than one year).

The resulting product reduces fatigue and has a refreshing and ergogenic effect.

An exemplary procedure for making the product follows:

Drone brood larvae are blended with a mixer (or any similar device) until homogeneous. 5 parts of glucose are added to the resulting drone brood homogenate. The product is thoroughly blended again with a mixer into a uniform mass. The resulting final product is then added to a powdered coffee substitute in a proportion of 1 to 9 and, once more, blended into a uniform mass. The product is used in accordance with the instructions for coffee substitute.

The inventive formulation of coffee substitute or other beverage follows:
Drone brood—1 part;
Glucose and/or fructose: from 3 to 9 parts; and
Fillers (a powdered coffee substitute or another beverage powder): from 1 to 100 parts.

Despite the fact that the ingredients of the claimed product are known, their combination in a single product is not known from the prior art, and namely the resulting formulation makes it possible to achieve the following objectives:
preserving and stabilizing the properties of drone brood,
providing the water solubility of the resulting product,
achieving the claimed technical result, namely, to enhance the preventive and therapeutic effect in health conditions associated with fatigue.

Therefore, the technical solution presents novelty and an inventive step.

1. A coffee substitute or other beverage intended for use in preventive and therapeutic treatment of health conditions associated with fatigue, consisting of:
drone brood—1 part;
glucose and/or fructose—3 to 9 parts;
fillers (powdered coffee substitute or other beverage powder)—1 to 100 parts.
